(19) United States
(12) Patent Application Publication
(10) Pub. No.: US 2007/0275052 A1
(43) Pub. Date: Nov. 29, 2007

(54) Pharmaceutical compositions containing sterol inhibitors

(75) Inventors: Abhay Mahajan, Khamgaon (IN); Kamal Mehta, Bhilwara (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Andheri (IN)

(21) Appl. No.: 11/805,752

(22) Filed: May 24, 2007

Related U.S. Application Data
(60) Provisional application No. 60/848,042, filed on Sep. 26, 2006.

(30) Foreign Application Priority Data
May 24, 2006 (IN) 790/MUM/2006

(51) Int. Cl. A61K 31/397; A61K 9/48; A61K 9/20; A61K 9/14

(52) U.S. Cl. 424/451; 424/464; 424/489; 514/210.02

(57) ABSTRACT

Pharmaceutical compositions containing ezetimibe or its pharmaceutically acceptable salt that is substantially free of microcrystalline cellulose or crystalline cellulose are disclosed. Also disclosed are pharmaceutical compositions containing micronized particles of ezetimibe.

### PRIORITY

This application claims the benefit of U.S. Provisional Application No. 60/848,042, filed on Sep. 26, 2006, and entitled “PHARMACEUTICAL COMPOSITIONS COMPRISING STEROL INHIBITORS”, and Indian Provisional Application No. 790/MUM/2006, filed on May 24, 2006, and entitled “PHARMACEUTICAL COMPOSITIONS COMPRISING STEROL INHIBITORS”.

### BACKGROUND OF THE INVENTION

1. Technical Field

The present invention generally relates to a pharmaceutical composition containing sterol inhibition inhibitors such as ezetimibe and pharmaceutically acceptable salts thereof and a process for its preparation.

2. Description of the Related Art

Ezetimibe is a selective cholesterol absorption inhibitor that effectively blocks intestinal absorption of dietary and biliary cholesterol and is represented by the structure of Formula I. 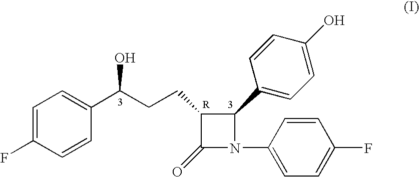
Ezetimibe undergoes glucuronidation to a single metabolite and is localized in the intestinal wall, where it prevents cholesterol absorption. Enterohepatic recirculation of ezetimibe and the glucoronide ensures repeated delivery to the site of action and limits peripheral exposure. Ezetimibe does not affect the absorption of fat-soluble vitamins or triglycerides. Ezetimibe is available under trade name ZETIA® marketed by Merck/Schering Plough (MSP Singapore).

U.S. Pat. Nos. 5,624,920, 5,656,624, 5,668,990, 5,688,787 and 5,767,115 disclose hydroxy-substituted azetidinone compounds and beta-lactam compounds useful for lowering cholesterol and/or in inhibiting the formation of cholesterol-containing lesions in mammalian arterial walls such as ezetimibe.

U.S. Pat. Nos. 5,661,145 and 5,846,966 disclose hydroxy-substituted azetidinone compounds and beta-lactam compounds in combination with HMG CoA reductase inhibitors for preventing or treating atherosclerosis and reducing plasma cholesterol levels.

WO00/38725 discloses cardiovascular therapeutic combinations including an ileal bile acid transport inhibitor or cholesteryl ester transport protein inhibitor in combination with a fibric acid derivative, nicotinic acid derivative, microsomal triglyceride transfer protein inhibitor, cholesterol absorption antagonist, phytosterol, stanol, antihypertensive agent or bile acid sequestrant.

U.S. Pat. No. 5,698,527 discloses ergostanone derivatives substituted with disaccharides as cholesterol absorption inhibitors, employed alone or in combination with certain other cholesterol lowering agents, which are useful in the treatment of hypercholesterolemia and related disorders.

U.S. Pat. No. 7,030,106 (“the 106” patent”) discloses compositions and therapeutic combinations comprising PPAR activator(s) and certain sterol absorption inhibitors for treating vascular and lipidemic conditions.

Heretofore, there has been no recognition or appreciation in the prior art of the effect of particle size on the dissolution profile of solid dosage forms such as tablets.

### SUMMARY OF THE INVENTION

In accordance with one embodiment of the present invention, a pharmaceutical composition comprising micronized particles of ezetimibe or a pharmaceutically acceptable salt thereof is provided wherein about 90% of the particles are not more than about 25 microns.

In accordance with a second embodiment of the present invention, a pharmaceutical composition comprising micronized particles of ezetimibe or a pharmaceutically acceptable salt thereof is provided wherein about 90% of the particles are not more than about 15 microns.

In accordance with a third embodiment of the present invention, a pharmaceutical composition comprising micronized particles of ezetimibe or a pharmaceutically acceptable salt thereof is provided wherein about 90% of the particles are not more than about 7 microns.

In accordance with a fourth embodiment of the present invention, a pharmaceutical composition comprising micronized particles of ezetimibe or a pharmaceutically acceptable salt thereof is provided wherein about 90% of the particles are not more than about 7 microns and about 50% of the particles are not more than about 5 microns.

In accordance with a fifth embodiment of the present invention, a pharmaceutical composition comprising ezetimibe or a pharmaceutically acceptable salt thereof is provided substantially free from microcrystalline cellulose or crystalline cellulose.

In accordance with the present invention, the particle size of ezetimibe required for adequate dissolution has now been discovered.

### DETAILED DESCRIPTION OF THE PREFERRED EMBODIMENTS

The present invention is directed to pharmaceutical compositions comprising ezetimibe or a pharmaceutically acceptable salt thereof which is substantially free from microcrystalline cellulose or crystalline cellulose. The present invention is further directed to pharmaceutical compositions comprising at least one sterol inhibitor or a pharmaceutically acceptable salt thereof such as ezetimibe having a specific particle size. Ezetimibe is well known and can be prepared according to known methods. In one embodiment, the pharmaceutical composition contains ezetimibe of which about 90% of the particles are not more than about 25 microns. In another embodiment, the pharmaceutical composition contains ezetimibe of which about 90% of the particles are not more than about 15 microns. In another embodiment, the pharmaceutical composition contains ezetimibe of which about 90% of the particles are not more than about 10 microns. In a most preferred embodiment about 90% of the particles are not more than about 7 microns. In one embodiment, the pharmaceutical composition contains ezetimibe of which about 50% of the particles are not more than about 10 microns. In another embodiment, the pharmaceutical composition contains ezetimibe of which about 50% of the particles are not more than about 7 microns. In another embodiment, the pharmaceutical composition contains ezetimibe of which about 50% of the particles are not more than about 4 microns.

The present invention is also directed to a process of making pharmaceutical compositions by direct compression and/or by a wet granulation process.

The pharmaceutical compositions of the present invention may contain one or more pharmaceutically acceptable excipients. Suitable pharmaceutically acceptable excipients include, but are not limited to, diluents, disintegrants, binders, lubricants and the like and mixtures thereof.

Suitable one or more diluents include lactose, dicalcium phosphate, calcium sulfate, kaolin, mannitol, sorbitol, inositol, sodium chloride, dry starch, powdered sugar and the like and mixtures thereof.

Suitable one or more disintegrants include carboxymethylcellulose calcium, carboxymethylcellulose sodium (e.g., Ac-Di-Sol®, Primellose croscarmellose sodium), sodium starch glycolate (e.g. Explotab®), crospovidone, guar gum, magnesium aluminum silicate, methyl cellulose, polacrilin potassium, powdered cellulose, pregelatinized starch, sodium alginate and starch, and the like and mixtures thereof wherein sodium starch glycolate is most preferred.

Suitable one or more binders include polyvinylpyrrolidone, starch mucilage, pregelatinized starch, sodium alginate, alginic acid, acacia mucilage, tragacanth, hydroxypropyl methyl cellulose, carboxymethylcellulose sodium, carboxymethylcellulose calcium, ethyl cellulose, polyethylene glycol, hydroxyethyl cellulose, hydroxypropyl cellulose, methyl cellulose, polymethacrylate, carboxyvinyl polymers such as carbopols and the like and mixtures thereof.

Suitable one or more lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate and the like and mixtures thereof.

The obtained pharmaceutical composition of ezetimibe was tested for its dissolution profile against Zetia® by using USP II apparatus at 100 rotations per minute (RPM).

### EXAMPLES

The following examples are intended to illustrate details of the invention, without thereby limiting it in any manner.

Initially a few experiments were carried out for ezetimibe having particle size about 25 microns which showed release of the drug about 40-50%, based on such release profile obtained ezetimibe was micronized and pharmaceutical dosage form was prepared. The example mentioned below demonstrates some illustrative procedures for preparing the pharmaceutical compositions described herein. The examples are provided to illustrate particular aspect of the disclosure and do not limit the scope of the present invention. The pharmaceutical compositions of present invention can be prepared with techniques well known in the art, preferably, direct compression, dry granulation and wet granulation.

### Example 1

Particle Size (D 0.9)=6 micron

TABLE I

| Ingredients | mg/tablet |
|---|---|
| Ezetimibe | 10 |
| Lactose monohydrate | 75 |
| Crospovidone | 5 |
| Sodium lauryl | 3 |
| sulphate | |
| Povidone K-30 | 6 |
| Purified water | q.s |
| Magnesium stearate | 1 |
| Total | 100 |

### Example 2

Particle Size (D 0.9)=6 micron

TABLE II

| Ingredients | mg/tablet |
|---|---|
| Ezetimibe | 10 |
| Lactose monohydrate | 75 |
| Sodium starch | 5 |
| glycolate | |
| Sodium lauryl | 3 |
| sulphate | |
| Povidone K-30 | 6 |
| Purified water | q.s |
| Magnesium stearate | 1 |
| Total | 100 |

Procedure

Ezetimibe, crospovidone or sodium starch glycolate, lactose monohydrate and sodium lauryl sulphate were sifted through ASTM mesh # 60. The pre granulation blend was transferred to the bowl of a rapid mixer granulator (RMG) followed by mixing for 10 minutes in RMG at slow impeller speed povidone K-30 in purified water was used as the binder solution for granulation; the powder blend was granulated at fast impeller and slow chopper to get desired granules. The granules were then dried in the Fluid Bed Drier till the loss on drying of the dried granules was found to be less than 3.0% w/w. The dried granules were then passed through ASTM mesh # 30 which was then mixed with magnesium stearate and blended in the Bin Blender for 3 minutes. The lubricated blend was compressed into tablets.

Comparative Dissolution Profile of Example 1, Example 2 and ZETIA®

Dissolution media (500 ml): water

Dissolution apparatus: USP II, 50 RPM

TABLE III

| TIME | EXAMPLE 1 | EXAMPLE 2 | ZETIA ® |
|---|---|---|---|
| (min) | (Percent Release) | (Percent Release) | (Percent Release) |
| 5 | 43 | 38 | 47 |
| 10 | 65 | 74 | 79 |
| 15 | 71 | 86 | 85 |
| 30 | 73 | 86 | 88 |
| 45 | 72 | 86 | 86 |
| 60 | 70 | 88 | 84 |
| Recovery | 75 | 95 | 81 |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A pharmaceutical composition comprising ezetimibe or a pharmaceutically acceptable salt thereof which is substantially free of microcrystalline cellulose or crystalline cellulose.

2. The pharmaceutical composition of claim 1, further comprising at least one excipient selected from the group consisting of a diluent, disintegrant, binder, lubricant and mixtures thereof.

3. The pharmaceutical composition of claim 2, wherein the diluent is selected from the group consisting of lactose, dicalcium phosphate, calcium sulfate, kaolin, mannitol, sorbitol, inositol, sodium chloride, dry starch, powdered sugar and mixtures thereof.

4. The pharmaceutical composition of claim 2, wherein the disintegrant is selected from the group consisting of carboxymethylcellulose calcium, carboxymethylcellulose sodium, sodium starch glycolate, crospovidone, guar gum, magnesium aluminum silicate, methyl cellulose, polacrilin potassium, powdered cellulose, pregelatinized starch, sodium alginate, starch and mixtures thereof.

5. The pharmaceutical composition of claim 2, wherein the binder is selected from the group consisting of polyvinylpyrrolidone, starch mucilage, pregelatinized starch, sodium alginate, alginic acid, acacia mucilage, tragacanth, hydroxypropyl methyl cellulose, carboxymethylcellulose sodium, carboxymethylcellulose calcium, ethyl cellulose, polyethylene glycol, hydroxyethyl cellulose, hydroxypropyl cellulose, methyl cellulose, polymethacrylate, carboxyvinyl polymers and mixtures thereof.

6. The pharmaceutical composition of claim 2, wherein the lubricant is selected from the group consisting of magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, zinc stearate and mixtures thereof.

7. The pharmaceutical composition of claim 1, comprising ezetimibe, lactose monohydrate, crospovidone, sodium lauryl sulphate, Povidone K-30 and magnesium stearate.

8. The pharmaceutical composition of claim 1, comprising ezetimibe, lactose monohydrate, sodium starch glycolate, sodium lauryl sulphate, Povidone K-30 and magnesium stearate.

9. The pharmaceutical composition of claim 1, in the form of a powder, tablet or a capsule.

10. A pharmaceutical composition comprising micronized particles of ezetimibe or a pharmaceutically acceptable salt thereof, wherein about 90% of the ezetimibe particles are not more than about 25 microns.

11. The pharmaceutical composition of claim 10, wherein about 90% of the ezetimibe particles are not more than about 15 microns.

12. The pharmaceutical composition of claim 10, wherein about 90% of the ezetimibe particles are not more than about 10 microns.

13. The pharmaceutical composition of claim 10, wherein about 90% of the ezetimibe particles are not more than about 7 microns.

14. The pharmaceutical composition of claim 10, wherein about 50% of the ezetimibe particles are not more than about 10 microns.

15. The pharmaceutical composition of claim 10, wherein about 50% of the ezetimibe particles are not more than about 7 microns.

16. The pharmaceutical composition of claim 10, wherein about 50% of the ezetimibe particles are not more than about 4 microns.

17. The pharmaceutical composition of claim 10, further comprising one or more pharmaceutically acceptable excipients.

18. The pharmaceutical composition of claim 10, which is substantially free of microcrystalline cellulose or crystalline cellulose.

19. The pharmaceutical composition of claim 10, in the form of a powder, tablet or a capsule.

20. A process for preparing a pharmaceutical composition, the process comprising (a) sifting ezetimibe and a pharmaceutically acceptable excipient comprising at least one of a diluent and a disintegrant through a sieve having openings less than, or equal to, about 60 mesh; (b) mixing the particles passing through the sieve; (c) granulating the mixture of (b) using a granulating solution comprising at least one binder; (d) drying the granules; (e) lubricating the granules; and (f) compressing the lubricated granules into tablets.
